(19) United States
(12) Patent Application Publication
(10) Pub. No.: US 2023/0310677 A1
(43) Pub. Date: Oct. 5, 2023

(54) STERILIZATION DEVICE AND METHOD BY COMBINING COLD PLASMA AND ULTRAVIOLET LIGHT

(71) Applicants: Nanjing Suman Plasma Engineering Research Institute Co., Ltd., Nanjing (CN); Nanjing Agricultural University, Nanjing (CN)

(72) Inventors: Jianhao ZHANG, Nanjing (CN); Jin WANG, Nanjing (CN); Xiaowei SHENG, Nanjing (CN); Long XU, Nanjing (CN); Jinglin WAN, Nanjing (CN); Xilin HOU, Nanjing (CN)

(21) Appl. No.: 17/953,287

(22) Filed: Sep. 26, 2022

Related U.S. Application Data
(63) Continuation-in-part of application No. PCT/CN2022/083528, filed on Mar. 29, 2022.

(30) Foreign Application Priority Data
Mar. 16, 2022 (CN) 202210259615.0
Mar. 25, 2022 (CN) 202210300336.4

(51) Int. Cl. A61L 2/14; A61L 2/26; A61L 9/20

(52) U.S. Cl. CPC A61L 2/14; A61L 2/26; A61L 9/20; A61L 2202/11; A61L 2202/15; A61L 2209/12

(57) ABSTRACT

A sterilizer includes a cold plasma generator; an ultraviolet (UV) light generator; and an airflow circulation system. The cold plasma generator and the UV light generator are disposed in the airflow circulation system. The cold plasma generator is configured to generate a cold plasma airflow; the UV light generator is configured to emit an UV light; the UV light generator is disposed in a downstream of the cold plasma airflow of the cold plasma generator, so that the cold plasma airflow is exposed to the UV light to form a disinfecting airflow that is then sprayed on a surface of a sample contaminated with a pathogen comprising coronavirus.

### CROSS-REFERENCE TO RELATED APPLICATIONS

This application is a continuation-in-part of International Patent Application No. PCT/CN2022/083528 with an international filing date of Mar. 29, 2022, designating the U.S., now pending, and further claims foreign priority benefits to Chinese Patent Application No. 202210259615.0 filed Mar. 16, 2022, and to Chinese Patent Application No. 202210300336.4 filed Mar. 25, 2022. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, MA 02142.

### BACKGROUND

The disclosure relates to the field of elimination of pathogens such as coronaviruses and, more particularly, to a sterilizer for pathogen elimination by using cold plasma and ultraviolet illumination.

Severe acute respiratory syndrome coronavirus 2 (SARS-CoV-2) has spread throughout the world and threatened human health and the economy. To stop the spread of coronaviruses, effective chemical and physical disinfection technologies have been developed. Chemical disinfection is a process in which pathogens are sprayed with or soaked in a disinfectant containing chlorine, peroxides, or other active ingredients. Cold chain is a system of transporting products in a low-temperature range. When the disinfectant is sprayed on the surface of the product, the surface temperature is cold enough to freeze the disinfectant, reducing the efficacy of disinfection. Furthermore, disinfectants typically contain certain chemicals that are harmful to human health, and are particularly harmful to practitioners of cold chain businesses.

### SUMMARY

The disclosure provides a sterilizer that comprises a cold plasma generator, an ultraviolet (UV) light generator, and an airflow circulation system. The cold plasma generator and the UV light generator are disposed in the airflow circulation system. The cold plasma generator is configured to generate a cold plasma airflow at 25° C.-35° C.; the UV light generator is configured to emit an UV light with a wavelength of 222 nm; the UV light generator is disposed in a downstream of the cold plasma airflow of the cold plasma generator, so that the cold plasma airflow is exposed to the UV light to form a disinfecting airflow that is then sprayed on a surface of a sample contaminated with a pathogen comprising coronavirus; and the airflow circulation system is configured to circulate the disinfecting airflow, thus increasing the disinfection efficiency of the sterilizer and reducing ozone emission.

In a class of this embodiment, the cold plasma generator comprises an electrode tube assembly, an electrode tube frame, a plurality of micro fans, and a micro fan frame; the electrode tube assembly comprises 2-5 layers of ceramic electrode tubes that are disposed in parallel or crosswise; a distance between every two adjacent ceramic electrode tubes is 6±2 mm; both ends of the ceramic electrode tubes are extended out of the electrode tube frame, and connected to two electrodes of a high-voltage power supply.

In a class of this embodiment, the UV light generator comprises 2-5 sets of UV light sources spaced evenly throughout the downstream of the cold plasma airflow of the cold plasma generator; the sample is disposed at 4-5 cm away from a centerline plane of the 2-5 sets of UV light sources; and the sample is disposed at 8-12 cm away from an air outlet of the cold plasma airflow.

In a class of this embodiment, the airflow circulation system comprises an axial flow fan, a pipeline, and a flow sensor; the flow sensor is connected to a programmable logic controller (PLC) to control the operation of the axial flow fan, thus regulating the flow rate of the cold plasma airflow.

In a class of this embodiment, the plurality of micro fans is spaced evenly on the micro fan frame; a horizontal plane of center points of the plurality of micro fans is 20±5 mm away from a horizontal plane of center points of a top ceramic electrode tube layer; and the plurality of micro fans is operated at a flow rate of 1-8 m³/min.

In a class of this embodiment, each of the ceramic electrode tubes comprises a ceramic shell, metal powders, and two silicone seals. The ceramic shell comprises aluminum oxide (Al₂O₃) of 99.5% by weight, an outer diameter is 15-25 mm, and a wall thickness is 1-3 mm; the two silicone seals are disposed on both ends of the ceramic shell, respectively; each silicone seal comprises silicon dioxide (SᵢO₂); and a copper or aluminum wire having a diameter of 2±0.5 mm is disposed through both central points of the two silicone seals.

In a class of this embodiment, the ceramic shell is filled with the metal powders comprising 30-60 parts of aluminum powder by mass, 30-60 parts of magnesium powder by mass, 10-30 parts of copper powder by mass, and 0.1-5 parts of titanium powder by mass; and the metal powders have a particle size of 80-150 mesh.

The disclosure also provides a method for eliminating a pathogen comprising coronavirus using the sterilizer, the method comprising: spraying the disinfecting airflow activated by the cold plasma airflow and the UV light on the surface of the sample contaminated with a pathogen comprising coronavirus for 30-150 seconds; and circulating the disinfecting airflow through the airflow circulation system thus increasing the disinfection efficiency of the sterilizer and reducing ozone emission.

In a class of this embodiment, the cold plasma generator works at an operating voltage of 10-40 kV, an operating frequency is 8-20 kHz, and a power density is 0.5-2.5 W/cm²; the UV light generator emits the UV light at a wavelength of 222±10 nm, and a power density is 0.25-0.65 W/cm².

The following advantages are associated with the cold plasma sterilizer of the disclosure.
(1) 222 nm UV light is a germicidal light capable of inactivating coronaviruses. The disclosure combines the 222 nm UV light with cold plasma technology to form a physical field that effectively kills the coronaviruses without harm to human health. The sterilizer of the disclosure is efficient, green and low of carbon in disinfecting the pathogens existed in large-scale and large-throughput automatic logistics.
(2) The sterilizer of the disclosure combines the properties of the cold plasma technology with those of the 222 nm UV light to achieve a higher level of germicidal activity, which provides greater effectiveness in killing pathogens such as the coronaviruses. The sterilizer is suitable for use in removing the coronaviruses from the specific scenarios such as cold chain logistics, express logistics, and arriving baggage.
(3) The disclosure optimizes the operating parameters of the cold plasma generator, such as the operating voltage, the operating frequency, and the flow rate of the, as well as the power density of the 222 nm UV light, so as to increase flow intensity, reduce energy consumption, and circulate the cold plasma airflow, thereby increasing the disinfection efficiency of the sterilizer and reducing ozone emission.

### DETAILED DESCRIPTION OF THE DRAWINGS

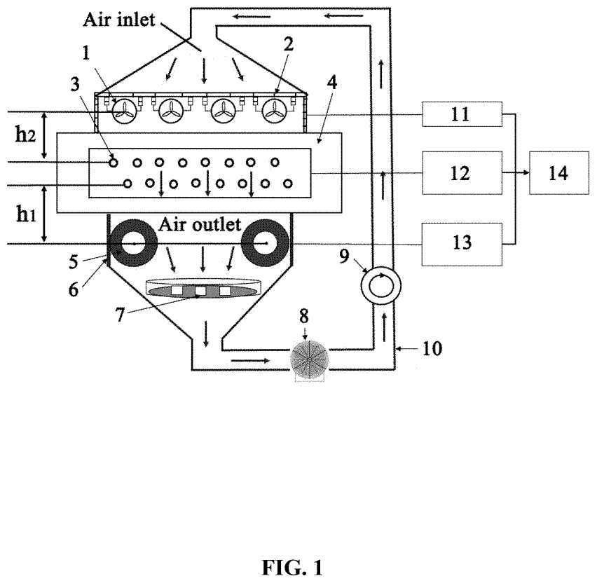

FIG. 1 is a schematic diagram of a sterilizer according to one example of the disclosure;

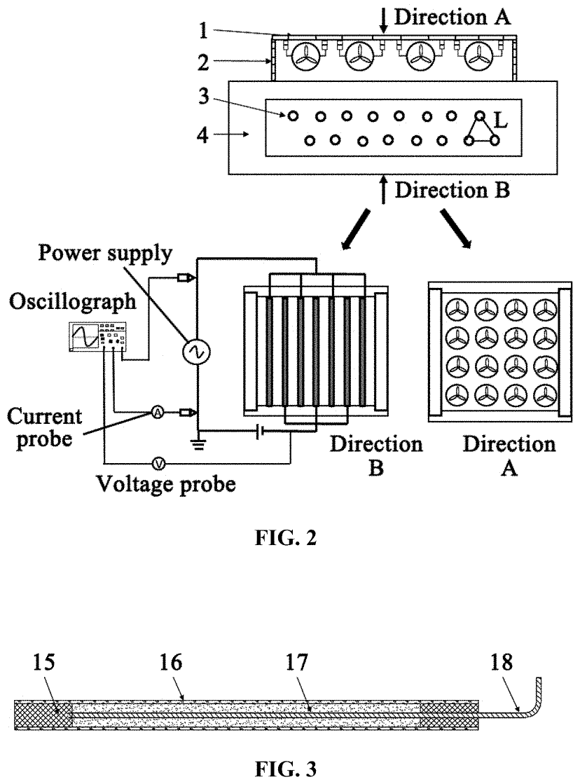

FIG. 2 is a schematic diagram of a cold plasma generator according to one example of the disclosure; and

FIG. 3 is a cross-sectional view of a ceramic electrode tube according to one example of the disclosure.

In the drawings, the following reference numbers are used: 1. Micro fan; 2. Micro fan frame; 3. Ceramic electrode tube; 4. Electrode tube frame; 5. UV light bulb; 6. UV light frame; 7. Sample; 8. Axial flow fan; 9. Flow sensor; 10. Pipeline; 11. First power supply; 12. Second power supply; 13. Third power supply; 14. Programmable logic controller; 15. Silicone seal; 16. Ceramic shell; 17. Metal powder; and 18. Wire.

### DETAILED DESCRIPTION OF THE EMBODIMENTS

As shown in FIGS. 1-3, the disclosure provides a sterilizer that comprises a cold plasma generator, an ultraviolet (UV) light generator, and an airflow circulation system. The cold plasma generator and the UV light generator are disposed in the airflow circulation system. The cold plasma generator is configured to generate a cold plasma airflow at 25° C.-35° C.; the UV light generator is configured to emit an UV light at a wavelength of 222 nm; the UV light generator is disposed in a downstream of the cold plasma airflow of the cold plasma generator, so that the cold plasma airflow is exposed to the UV light to form a disinfecting airflow that is then sprayed on a surface of a sample 7 contaminated with a pathogen comprising coronavirus; and the airflow circulation system is configured to circulate the disinfecting airflow, thus increasing the disinfection efficiency of the sterilizer and reducing ozone emission.

The cold plasma generator comprises an electrode tube assembly, an electrode tube frame 4, a plurality of micro fans 1, and a micro fan frame 2; the electrode tube assembly comprises two layers of ceramic electrode tubes 3 that are disposed in parallel or cross each other; each layer of ceramic electrode tubes comprises eight ceramic electrode tubes; a distance between every two adjacent ceramic electrode tubes is 6±2 mm; both ends of the ceramic electrode tubes are extended out of the electrode tube frame, and respectively connected to two electrodes of a high-voltage power supply.

The UV light generator comprises two sets of UV light bulbs 5 and two UV light frames 6 which spaced evenly throughout the downstream of the cold plasma airflow of the cold plasma generator. The airflow circulation system comprises an axial flow fan 8, a pipeline 10, and a flow sensor 9; the flow sensor is connected to a programmable logic controller (PLC) 14 so as to control the operation of the axial flow fan, thus regulating the cold plasma airflow. The sterilizer further comprises a power supply comprising a first power supply 11, a second power supply 12, and a third power supply 13. The first power supply 11 is configured to offer electric power to the flow fan; the second power supply 12 is configured to offer electric power to the cold plasma; and the third power supply 13 is configured to offer electric power to the UV light.

The plurality of micro fans is spaced evenly on the micro fan frame; a horizontal plane through center points of the plurality of micro fans is disposed at a height h₂ of 22 mm from a horizontal plane through center points of a top layer of the ceramic electrode tubes; and the plurality of micro fans is operated at a flow rate of 4-5 m³/min.

Each of the ceramic electrode tubes comprises a ceramic shell 16, metal powders 17, and two silicone seals 15. The ceramic shell comprises aluminum oxide (Al₂O₃) of 99.5% by weight, an outer diameter of 20 mm, and a wall thickness of 2 mm; the two silicone seals are disposed on both ends of the ceramic shell; each silicone seal comprises silicon dioxide (SᵢO₂); and a copper or aluminum wire 18 having a diameter of 2±0.5 mm is disposed through both central points of the two silicone seals; the ceramic shell is filled with the metal powders comprising 40 parts of aluminum powder by mass, 40 parts of magnesium powder by mass, 19 parts of copper powder by mass, and 1 part of titanium powder by mass; and the metal powders have a particle size of 100-120 mesh; and the electrode tube frame and the micro fan frame both comprise polytetrafluoroethylene.

The cold plasma airflow is exposed to the UV light to form a disinfecting airflow that is then sprayed onto the surface of the sample, so that the pathogens, such as the coronaviruses, are killed in 30-150 seconds; and the airflow circulation system is configured to circulate the cold plasma airflow, thus increasing the disinfection efficiency of the sterilizer and reducing ozone emission.

To further illustrate the disclosure, embodiments detailing a sterilizer are described below. It should be noted that the following embodiments are intended to describe and not to limit the disclosure.

### Example 1

Effect of operating frequency of high-voltage electric field on staphylococcus aureus.

10 μL of 10⁸ CFU/mL staphylococcus aureus (Gram-positive bacterium) suspension was aspirated, dropped onto a piece of quartz glass (defatted and sterilized) of size 1 cm×1 cm, spread evenly over a 1 cm² area, and dried at room temperature to form a sample. Several groups of samples were prepared, placed at a distance of 9±0.5 cm from the air outlet of the cold plasma airflow, kept at a height of 4-5 cm from a centerline plane of the two sets of UV light sources, and underwent the following disinfection processes, respectively: 1) cold plasma airflow for 30 s and 222 nm UV light for 30 s; 2) 222 nm UV light for 30 s and cold plasma airflow for 30 s; 3) cold plasma airflow for 60 s and 222 nm UV light for 60 s; 4) UV light at 222 nm for 60 s and cold plasma airflow for 60 s; 5) cold plasma airflow exposed to 222 nm UV light for 30 s (e.g. treated with the sterilizer of the disclosure); or 6) cold plasma airflow exposed to 222 nm UV light for 60 s (e.g. treated with the sterilizer of the disclosure); and the several groups of samples were marked as CP+UV-1, UV+CP-1, CP+UV-2, UV+CP-2, UV-CP-1, and UV-CP-2, respectively. A control group was an untreated comparison group. In treatment groups, the cold plasma generator worked at an operating voltage of 33±5 kV, and a high-voltage electric field had an operating frequency of 8.5 kHz and 19 kHz. Operating parameters: the power density of the cold plasma generator was 2.5 W/cm²; the UV light generator emitted a UV light at a wavelength of 222 nm, and a power density was 0.6 W/cm². The electrode tube assembly comprised two layers of ceramic electrode tubes that were disposed in parallel. A plate count method was used to measure the antibacterial activity of the different treatments.

TABLE 1
Effect of operating frequency of high-voltage electric field on staphylococcus aureus

| Operating | Staphylococcus aureus (Log10 CFU/cm²) | | | | | | |
|---|---|---|---|---|---|---|---|
| frequency | control | GW − UV-1 | UV + CP-1 | CP + UV-2 | UV + CP-2 | UV − CP-1 | UV − CP-2 |
| 8.5 | 6.41 ± 0.12 | 4.92 ± 0.04 | 4.87 ± 0.21 | 2.68 ± 0.07 | 2.54 ± 0.11 | 4.21 ± 0.14 | 1.78 ± 0.23 |
| 19 | | 4.97 ± 0.11 | 4.79 ± 0.15 | 2.56 ± 0.13 | 2.51 ± 0.16 | 4.24 ± 0.07 | 1.83 ± 0.12 |

As shown in Table 1, the staphylococcus aureus counts in the control group were 6.41±0.12 Log₁₀ CFU/cm²; the UV-CP-1 treatment group reduced the staphylococcus aureus counts compared with the CP+UV-1 treatment group and the UV+CP-1 treatment group; the UV-CP-2 treatment group reduced the staphylococcus aureus counts compared with the CP+UV-2 treatment group and the UV+CP-2 treatment group; the results showed that a simultaneous treatment achieved a greater efficiency of disinfection than a sequential treatment; when the high-voltage electric field had an operating frequency of 8.5 kHz and 19 kHz, the staphylococcus aureus counts in the UV-CP-2 treatment group were reduced to 1.78±0.23 and 1.83±0.12 Log₁₀ CFU/cm², which were less than the original number by 4.63 and 4.58 Log₁₀ CFU/cm², respectively, illustrating that there were no significant differences (p>0.05) between 8.5 kHz treatment groups and 19 kHz treatment group. Preferably, the following examples were implemented at the operating frequency of 8.5 kHz.

### Example 2

Effect of power density of cold plasma generator on staphylococcus aureus.

10 μL of 10⁸ CFU/mL staphylococcus aureus (Gram-positive bacterium) suspension was aspirated, dropped onto a piece of quartz glass (defatted and sterilized) of size 1 cm×1 cm, spread evenly over a 1 cm² area, and dried at room temperature to form a sample. Several groups of samples were prepared, placed at a distance of 9±0.5 cm from the air outlet of the cold plasma airflow, kept at a height of 4-5 cm from a centerline plane of the two sets of UV light sources, and underwent the following disinfection processes, respectively: 1) cold plasma airflow for 30 s and 222 nm UV light for 30 s; 2) 222 nm UV light for 30 s and cold plasma airflow for 30 s; 3) cold plasma airflow for 60 s and 222 nm UV light for 60 s; 4) UV light at 222 nm for 60 s and cold plasma airflow for 60 s; 5) cold plasma airflow exposed to 222 nm UV light for 30 s (e.g. treated with the sterilizer of the disclosure); or 6) cold plasma airflow exposed to 222 nm UV light for 60 s (e.g. treated with the sterilizer of the disclosure); and the Several groups of samples were marked as CP+UV-1, UV+CP-1, CP+UV-2, UV+CP-2, UV-CP-1, and UV-CP-2, respectively. A control group was an untreated comparison group. In treatment groups, a power density of the cold plasma generator was 0.5, 1.0, 1.5, 2.0 and 2.5 W/cm². Operating parameters: the cold plasma generator worked at an operating voltage of 33±5 kV, and a high-frequency electric field had an operating frequency of 8.5 kHz; the UV light generator emitted a UV light at a wavelength of 222 nm, and a power density was 0.6 W/cm². The electrode tube assembly comprised two layers of ceramic electrode tubes that were disposed in parallel. A plate count method was used to measure the antibacterial activity of the different treatments.

TABLE 2
Effect of power density of cold plasma generator on staphylococcus aureus

| Power | | | | | | | |
|---|---|---|---|---|---|---|---|
| density | Staphylococcus aureus (Log10 CFU/cm²) | | | | | | |
| (W/cm²) | control | CP + UV-1 | UV + CP-1 | CP + UV-2 | UV + CP-2 | UV − CP-1 | UV − CP-2 |
| 0.5 | 6.41 ± 0.07 | 5.50 ± 0.10 | 5.56 ± 0.06 | 2.79 ± 0.14 | 2.68 ± 0.09 | 4.95 ± 0.03 | 2.47 ± 0.33 |
| 1.0 | | 5.34 ± 0.03 | 5.38 ± 0.08 | 2.65 ± 0.08 | 2.60 ± 0.18 | 4.71 ± 0.06 | 2.35 ± 0.24 |
| 1.5 | | 5.13 ± 0.09 | 5.21 ± 0.17 | 2.46 ± 0.27 | 2.49 ± 0.15 | 4.54 ± 0.14 | 1.73 ± 0.16 |
| 2.0 | | 4.97 ± 0.17 | 4.95 ± 0.22 | 2.41 ± 0.07 | 2.53 ± 0.21 | 4.38 ± 0.13 | 1.47 ± 0.04 |
| 2.5 | | 4.88 ± 0.07 | 4.82 ± 0.05 | 2.32 ± 0.13 | 2.27 ± 0.23 | 4.15 ± 0.04 | 1.09 ± 0.07 |

As shown in Table 2, the staphylococcus aureus counts in the control group were 6.41±0.07 Log₁₀ CFU/cm²; the UV-CP-1 treatment group reduced the staphylococcus aureus counts compared with the CP+UV-1 treatment group and the UV+CP-1 treatment group; the UV-CP-2 treatment group reduced the staphylococcus aureus counts compared with the CP+UV-2 treatment group and the UV+CP-2 treatment group; the results showed that a simultaneous treatment achieved a greater efficiency of disinfection than a sequential treatment; when the power density of the cold plasma generator was 0.5, 1.0, 1.5, 2.0 and 2.5 W/cm², the staphylococcus aureus counts were 2.47±0.34, 2.35±0.24, 1.73±0.16, 1.47±0.04, and 1.09±0.10 Log₁₀ CFU/cm², respectively, which were less than the original number by 3.94, 4.05, 4.67, 4.93 and 5.31 Log₁₀ CFU/cm²; and the results showed that the disinfection effect increased with increasing power density. Preferably, in the following examples, the power density of the cold plasma was 2.5 W/cm².

### Example 3

Effect of power density of UV light generator on staphylococcus aureus.

10 μL of 10⁸ CFU/mL staphylococcus aureus (Gram-positive bacterium) suspension was aspirated, dropped onto a piece of quartz glass (defatted and sterilized) of size 1 cm×1 cm, spread evenly over a 1 cm² area, and dried at room temperature to form a sample. Several groups of samples were prepared, placed at a distance of 9±0.5 cm from the air outlet of the cold plasma airflow, kept at a height of 4-5 cm from a centerline plane of the 2 sets of UV light sources, and underwent the following disinfection processes, respectively: 1) cold plasma airflow for 30 s and 222 nm UV light for 30 s; 2) 222 nm UV light for 30 s and cold plasma airflow for 30 s; 3) cold plasma airflow for 60 s and 222 nm UV light for 60 s; 4) UV light at 222 nm for 60 s and cold plasma airflow for 60 s; 5) cold plasma airflow exposed to 222 nm UV light for 30 s (e.g. treated with the sterilizer of the disclosure); or 6) cold plasma airflow exposed to 222 nm UV light for 60 s (e.g. treated with the sterilizer of the disclosure); and the Several groups of samples were marked as CP+UV-1, UV+CP-1, CP+UV-2, UV+CP-2, UV-CP-1, and UV-CP-2, respectively. A control group was an untreated comparison group. In treatment groups, the UV light generator emitted a UV light at a wavelength of 222 nm, and a power density was 0.2, 0.4 and 0.6 W/cm². Operating processes: the cold plasma generator worked at an operating voltage of 33±5 kV, and a high-voltage electric field has an operating frequency of 8.5 kHz, and a power density was 2.5 W/cm². The electrode tube assembly comprised two layers of ceramic electrode tubes that were disposed in parallel. A plate count method was used to measure the antibacterial activity of the different treatments.

TABLE 3
Effect of power density of UV light generator on staphylococcus aureus

| Power | | | | | | | |
|---|---|---|---|---|---|---|---|
| density of | Staphylococcus aureus (Log10 CFU/cm²) | | | | | | |
| UV light | control | CP + UV-1 | UV + CP-1 | CP + UV-2 | UV + CP-2 | UV − CP-1 | UV − CP-2 |
| 0.2 | 6.38 ± 0.32 | 5.39 ± 0.07 | 5.42 ± 0.11 | 3.07 ± 0.03 | 3.13 ± 0.16 | 4.67 ± 0.05 | 2.24 ± 0.14 |
| 0.4 | | 5.08 ± 0.04 | 5.06 ± 0.16 | 2.88 ± 0.14 | 2.96 ± 0.19 | 4.48 ± 0.16 | 2.18 ± 0.06 |
| 0.6 | | 4.91 ± 0.14 | 4.89 ± 0.21 | 2.46 ± 0.25 | 2.51 ± 0.06 | 4.19 ± 0.08 | 1.76 ± 0.05 |

As shown in Table 3, the staphylococcus aureus counts in the control group were 6.38±0.32 Log₁₀ CFU/cm²; the UV-CP-1 treatment group reduced the staphylococcus aureus counts compared with the CP+UV-1 treatment group and the UV+CP-1 treatment group; the UV-CP-2 treatment group reduced the staphylococcus aureus counts compared with the CP+UV-2 treatment group and the UV+CP-2 treatment group; the results showed that a simultaneous treatment achieved a greater efficiency of sterilization than a sequential treatment; when the power density of the UV light generator was 0.2, 0.4 and 0.6 W/cm², the staphylococcus aureus counts in UV+CP treatment groups were 2.24±0.14, 2.18±0.06 and 1.76±0.05 Log₁₀ CFU/cm², respectively, which were less than the original number by 4.14, 4.20 and 4.62 Log₁₀ CFU/cm²; and the results showed that there was no significant difference (p>0.05) between the power densities of 0.2 and 0.4 W/cm², but they were significantly different from the power densities of 0.6 W/cm² (p<0.05). Preferably, in the following examples, the power density of the UV light was 0.6 W/cm².

### Example 4

Effect of number of layers of ceramic electrode tubes in cold plasma sterilizer on staphylococcus aureus.

10 μL of 10⁸ CFU/mL staphylococcus aureus (Gram-positive bacterium) suspension was aspirated, dropped onto a piece of quartz glass (defatted and sterilized) of size 1 cm×1 cm, spread evenly over a 1 cm² area, and dried at room temperature to form a sample. Several groups of samples were prepared, placed at a distance of 9±0.5 cm from the air outlet of the cold plasma airflow, kept at a height of 4-5 cm from a centerline plane of the 2 sets of UV light sources, and underwent the following disinfection processes, respectively: 1) cold plasma airflow for 30 s and 222 nm UV light for 30 s; 2) 222 nm UV light for 30 s and cold plasma airflow for 30 s; 3) cold plasma airflow for 60 s and 222 nm UV light for 60 s; 4) UV light at 222 nm for 60 s and cold plasma airflow for 60 s; 5) cold plasma airflow exposed to 222 nm UV light for 30 s (e.g. treated with the sterilizer of the disclosure); or 6) cold plasma airflow exposed to 222 nm UV light for 60 s (e.g. treated with the sterilizer of the disclosure); and the Several groups of samples were marked as CP+UV-1, UV+CP-1, CP+UV-2, UV+CP-2, UV-CP-1, and UV-CP-2, respectively. A control group was an untreated comparison group. In treatment groups, the electrode tube assembly comprised one, two, and three layers of the ceramic electrode tubes. Operational parameters: the cold plasma generator worked at an operating voltage of 33±5 kV, a high-frequency electric field had a frequency of 8.5 kHz, and a power density was 2.5 W/cm²; the UV light generator emitted a UV light at a wavelength of 222±10 nm, and a power density was 0.6 W/cm². The electrode tube assembly comprised two layers of ceramic electrode tubes that were disposed in parallel. A plate count method was used to measure the antibacterial activity of the different treatments.

TABLE 4
Effect of number of layers of ceramic electrode tubes on staphylococcus aureus

| Number of layers | | | | | | | |
|---|---|---|---|---|---|---|---|
| of ceramic | Staphylococcus aureus (Log10 CFU/cm²) | | | | | | |
| electrode tubes | control | CP + UV-1 | UV + CP-1 | CP + UV-2 | UV + CP-2 | UV − CP-1 | UV − CP-2 |
| 1 | 6.50 ± 0.19 | 5.63 ± 0.11 | 5.70 ± 0.18 | 2.59 ± 0.07 | 2.60 ± 0.12 | 5.08 ± 0.05 | 2.45 ± 0.14 |
| 2 | | 4.76 ± 0.06 | 4.81 ± 0.13 | 2.47 ± 0.15 | 2.51 ± 0.06 | 4.21 ± 0.16 | 1.65 ± 0.13 |
| 3 | | 5.15 ± 0.14 | 5.23 ± 0.16 | 2.65 ± 0.02 | 2.63 ± 0.08 | 4.64 ± 0.08 | 2.080.17 |

As shown in Table 4, the staphylococcus aureus counts in the control group were 6.50±0.19 Log₁₀ CFU/cm²; the UV-CP-1 treatment group reduced the staphylococcus aureus counts compared with the CP+UV-1 treatment group and the UV+CP-1 treatment group; the UV-CP-2 treatment group reduced the staphylococcus aureus counts compared with the CP+UV-2 treatment group and the UV+CP-2 treatment group; the results showed that a simultaneous treatment achieved a greater efficiency of sterilization than a sequential treatment. when the electrode tube assembly In UV+CP treatment groups comprised one, two, and three layers of the ceramic electrode tubes, the staphylococcus aureus counts were 2.45±0.09, 1.65±0.13, and 2.08±0.17 Log₁₀ CFU/cm², respectively, which were less than the original number by 4.05, 4.85 and 4.42 Log₁₀ CFU/cm²; and the results showed that there was significant difference (p>0.05) in the staphylococcus aureus counts between the treatment groups difference in layers of the ceramic electrode tubes. Preferably, the following examples were implemented by using two layers of the ceramic electrode tubes.

### Example 5

Effect of types of layers of ceramic electrode tubes on staphylococcus aureus.

10 μL of 10⁸ CFU/mL staphylococcus aureus (Gram-positive bacterium) suspension was aspirated, dropped onto a piece of quartz glass (defatted and sterilized) of size 1 cm×1 cm, spread evenly over a 1 cm² area, and dried at room temperature to form a sample. Several groups of samples were prepared, placed at a distance of 9±0.5 cm from the air outlet of the cold plasma airflow, kept at a height of 4-5 cm from a centerline plane of the 2 sets of UV light sources, and underwent the following disinfection processes, respectively: 1) cold plasma airflow for 30 s and 222 nm UV light for 30 s; 2) 222 nm UV light for 30 s and cold plasma airflow for 30 s; 3) cold plasma airflow for 60 s and 222 nm UV light for 60 s; 4) UV light at 222 nm for 60 s and cold plasma airflow for 60 s; 5) cold plasma airflow exposed to 222 nm UV light for 30 s (e.g. treated with the sterilizer of the disclosure); or 6) cold plasma airflow exposed to 222 nm UV light for 60 s (e.g. treated with the sterilizer of the disclosure); and the Several groups of samples were marked as CP+UV-1, UV+CP-1, CP+UV-2, UV+CP-2, UV-CP-1, and UV-CP-2, respectively. A control group was an untreated comparison group. In treatment groups, the layers of the ceramic electrode tube were disposed in parallel or crosswise. Operating parameters: the cold plasma generator worked at an operating voltage of 33±5 kV, a high-frequency electric field had a frequency of 8.5 kHz, and a power density was 2.5 W/cm²; the UV light generator emitted a UV light at a wavelength of 222 nm, a power density was 0.6 W/cm²; and the airflow circulation system circulated the cold plasma airflow at a flow rate of 4-6 m³/min. The electrode tube assembly comprised two layers of ceramic electrode tubes. A plate count method was used to measure the antibacterial activity of the different treatments.

TABLE 5
Effect of types of layers of ceramic electrode tubes on staphylococcus aureus

Types of layers

| of ceramic | Staphylococcus aureus (Log10 CFU/cm²) | | | | | | |
|---|---|---|---|---|---|---|---|
| electrode tubes | control | CP + UV-1 | UV + CP-1 | CP + UV-2 | UV + CP-2 | UV − CP-1 | UV − CP-2 |
| Parallel | 6.83 ± 0.24 | 5.81 ± 0.12 | 5.73 ± 0.24 | 2.69 ± 0.06 | 2.62 ± 0.15 | 4.31 ± 0.14 | 1.32 ± 0.16 |
| Crossed | | 5.39 ± 0.09 | 5.42 ± 0.17 | 2.51 ± 0.14 | 2.43 ± 0.21 | 4.82 ± 0.05 | 1.83 ± 0.15 |

As shown in Table 5, the staphylococcus aureus counts in the control group were 6.83±0.24 Log₁₀ CFU/cm²; the UV-CP-1 treatment group reduced the staphylococcus aureus counts compared with the CP+UV-1 treatment group and the UV+CP-1 treatment group; the UV-CP-2 treatment group reduced the staphylococcus aureus counts compared with the CP+UV-2 treatment group and the UV+CP-2 treatment group; the results showed that a simultaneous treatment achieved a greater efficiency of sterilization than a sequential treatment; when the layers of the ceramic electrode tubes in the UV+CP treatment groups were disposed in parallel or crosswise, the staphylococcus aureus counts in UV+CP treatment groups were 1.32±0.16 and 1.83±0.15 Log₁₀ CFU/cm², respectively, which were less than the original number by 5.51 and 5.00 Log₁₀ CFU/cm²; and the results showed that there was significant difference (p<0.05) in the staphylococcus aureus counts between the treatment groups which were different in types of layers of the ceramic electrode tubes. Preferably, in the following examples, the layers of the ceramic electrode tubes were disposed in parallel.

### Example 6

Effect of disinfection time on staphylococcus aureus.

10 μL of 10⁸ CFU/mL staphylococcus aureus (Gram-positive bacterium) suspension was aspirated, dropped onto a piece of quartz glass (defatted and sterilized) of size 1 cm×1 cm, spread evenly over a 1 cm² area, and dried at room temperature to form a sample. Several groups of samples were prepared and separately placed into a petri dish, and treated with a cold plasma airflow and 222 nm UV light for different seconds; the petri dish was placed in placed at a distance of 9±0.5 cm from the air outlet of the cold plasma airflow, and kept at a height of 4.3±0.2 cm from a centerline plane of the two sets of UV light sources. A control group was an untreated comparison group. In treatment groups, the several groups of samples were treated for 20 s, 40 s, 60 s, 80 s, 100 s, 120 s, 140 s and 160 s, respectively. Operating parameters: the cold plasma generator worked at an operating voltage of 33±5 kV, a high-frequency electric field had a frequency of 8.5 kHz, and a power density was 2.5 W/cm²; the UV light generator emitted a UV light at a wavelength of 222 nm, and a power density was 0.6 W/cm²; and the electrode tube assembly comprised two layers of ceramic electrode tubes that were disposed in parallel. A plate count method was used to measure the antibacterial activity of the different treatment groups. A thermometer was used to measure the surface temperature of each sample disinfected for different seconds.

TABLE 6
Effect of disinfection time on staphylococcus aureus

| Disinfection | Staphylococcus aureus | Disinfection | Surface |
|---|---|---|---|
| time | (Log10 CFU/cm²) | rate (%) | temperature (° C.) |
| Control | 6.47 ± 0.06 | — | 22.4 |
| 20 | 5.69 ± 0.07 | 80.7478 | 24.5 |
| 40 | 3.54 ± 0.26 | 99.8643 | 25.5 |
| 60 | 1.93 ± 0.06 | 99.9967 | 27.1 |
| 80 | 1.83 ± 0.07 | 99.9973 | 28.7 |
| 100 | 1.50 ± 0.15 | 99.9988 | 29.4 |
| 120 | 1.26 ± 0.06 | 99.9993 | 30.6 |
| 140 | 0.79 ± 0.33 | 99.9998 | 31.5 |
| 160 | 0 | 100.0000 | 32.5 |

As shown in Table 6, the staphylococcus aureus counts in the control group were 6.47±0.06 Log₁₀ CFU/cm²; after disinfection for 20 s, 40 s, 60 s, 80 s, 100 s, 120 s and 140 s, the treatment groups reduced the staphylococcus aureus counts to 5.69±0.06, 3.54±0.26, 1.93±0.06, 1.83±0.07, 1.50±0.15, 1.26±0.06 and 0.79±0.33 Log₁₀ CFU/cm², respectively, which were less than the original number by 0.78, 2.93, 4.54, 4.63, 4.97, 5.20 and 5.68 Log₁₀ CFU/cm²; and staphylococcus aureus was killed after disinfection for 160 s. The results showed that sterilization effect increased with increasing disinfection time; as the sterilization time increased, the surface temperature of each sample increased but remained below 34° C.

### Example 7

Sterilization of killing SARS-CoV-2 by cold plasma and 222 nm UV light.

SARS-CoV-2 was used as a target pathogen and disinfected under the preferred conditions tested in FIGS. 1-6.

At a temperature of 20° C. and 50% relative humidity, 10 μL of 10⁷ TCID 50/mL coronavirus solution (SARS-CoV-2: BetaCoV/JS02/Human/2019) was aspirated, dropped onto a piece of quartz glass (defatted and sterilized) of size 1 cm×1 cm, spread evenly over a 1 cm² area, and dried at room temperature to form a sample. Several groups of samples were prepared, separately placed into a petri dish, and treated with a cold plasma airflow and 222 nm UV light for different seconds; each petri dish was placed in placed at a distance of 9±0.5 cm from the air outlet of the cold plasma airflow, and kept at a height of 4.3±0.2 cm from a centerline plane of the two sets of UV light sources. Two control groups were untreated comparison groups and included a positive control group (exposed to the pathogen) and a negative control group (exposed to cells). In treatment groups, the several groups of samples were treated for 20 s, 40 s, 60 s, 80 s, 100 s, 120 s, 140 s and 160 s, respectively. Operating parameters: the cold plasma generator worked at an operating voltage of 33±5 kV, a high-voltage electric field has an operating frequency of 8.5 kHz, and a power density was 2.5 W/cm²; the UV light generator emitted a UV light at a wavelength of 222 nm, a power density was 0.6 W/cm²; the electrode tube assembly comprised two layers of ceramic electrode tubes that were disposed in parallel. The Median Tissue Culture Infectious Dose (TCID₅₀) assay was used to quantify a virus titer of SARS-CoV-2 (The experimental materials were provided by the Jiangsu Provincial Center for Disease Control and Prevention, and the experiment was carried out in the P3 laboratory of the Jiangsu Provincial Center for Disease Control and Prevention). A thermometer was used to measure the surface temperature of each sample disinfected for different seconds. An ozone detector was used to measure an ozone concentration. Operating parameters: a high-frequency electric field had a frequency of 8.5 kHz, and a power density of the cold plasma generator was 2.5 W/cm²; the UV light generator emitted a UV light at a wavelength of 222 nm, and a power density was 0.6 W/cm²; the electrode tube assembly comprised two layers of ceramic electrode tubes that were disposed in parallel and disinfected for 60 s.

TABLE 7
Changes in surface temperature and ozone concentration

| Sterilization | Ozone | Surface |
|---|---|---|
| time (s) | concentration (ppm) | temperature (° C.) |
| 0 | 1100 | 19.1 |
| 30 | 1090 | 22.1 |
| 60 | 1020 | 24.5 |
| 90 | 1000 | 26.1 |
| 120 | 1112 | 29.3 |
| 150 | 1077 | 29.8 |

TABLE 8
Sterilization for killing SARS-CoV-2

| | Virus titer | Disinfection |
|---|---|---|
| Group | (log₁₀TCID₅₀/cm²) | rate (%) |
| Positive control group | 4.28 | — |
| Negative control group | 0 | — |
| 30 s | 0 | 100 |
| 60 s | 0 | 100 |
| 90 s | 0 | 100 |
| 120 s | 0 | 100 |
| 150 s | 0 | 100 |

As shown in Table 7 and Table 8, the several groups of samples were sterilized for 30 s, 60 s, 90 s, 120 s and 150 s, respectively, and all coronavirus was killed. The cells in the negative control group grew normally; the logarithmic value of average virus titer of SARS-CoV-2 in the positive control group was 4.28, ranging between 4.00 and 4.50; and the growth of the cells in the treatment group was consistent with that of the negative control group. The surface temperature of each sample increased with the disinfection time; after disinfection for 150 s, the surface temperature was remained below 30° C. and the ozone concentration was maintained at 1000-1100 ppm. The results showed that the sterilizer of the disclosure killed SARS-CoV-2 by the use of the cold plasma airflow whose properties were not affected by a temperature rise due to UV radiation.

The disclosure revealed that at a temperature of 20° C. and 50% relative humidity, the samples were placed at a distance of 9±0.5 cm from the air outlet of the cold plasma airflow, kept at a height of 4-5 cm from a centerline plane of the two sets of UV light sources, and disinfected for 30 s, so that all SARS-CoV-2 was destroyed, thus meeting the requirements of the disinfection standard.

It will be obvious to those skilled in the art that changes and modifications may be made, and therefore, the aim in the appended claims is to cover all such changes and modifications.

What is claimed is:

1. A sterilizer, comprising:
1) a cold plasma generator;
2) an ultraviolet (UV) light generator; and
3) an airflow circulation system;
wherein
the cold plasma generator and the UV light generator are disposed in the airflow circulation system; the cold plasma generator is configured to generate a cold plasma airflow at 25° C.-35° C.; the UV light generator is configured to emit a 222 nm UV light; the UV light generator is disposed in a downstream of the cold plasma airflow of the cold plasma generator, so that the cold plasma airflow is exposed to the UV light to form a disinfecting airflow that is sprayed on a surface of a sample contaminated with a pathogen comprising coronavirus.

2. The sterilizer of claim 1, wherein the cold plasma generator comprises an electrode tube assembly, an electrode tube frame, a plurality of micro fans, and a micro fan frame; the electrode tube assembly comprises 2-5 layers of ceramic electrode tubes that are disposed in parallel or crosswise; a distance between every two adjacent ceramic electrode tubes is 6±2 mm; both ends of the ceramic electrode tubes are extended out of the electrode tube frame, and respectively connected to two electrodes of a high-voltage power supply.

3. The sterilizer of claim 1, wherein the UV light generator comprises 2-5 sets of UV light sources spaced evenly throughout the downstream of the cold plasma airflow of the cold plasma generator; the sample is disposed at 4-5 cm away from a centerline plane of the 2-5 sets of UV light sources; and the sample is disposed at 8-12 cm away from an air outlet of the cold plasma airflow.

4. The sterilizer of claim 3, wherein the airflow circulation system comprises an axial flow fan, a pipeline, and a flow sensor; the flow sensor is connected to a programmable logic controller (PLC) to control the operation of the axial flow fan, thus regulating a flow rate of the cold plasma airflow.

5. The sterilizer of claim 2, wherein the plurality of micro fans is spaced evenly on the micro fan frame; a horizontal plane of center points of the plurality of micro fans away from a horizontal plane of center points of a top ceramic electrode tube layer (h₂) is 20±5 mm; and the plurality of micro fans is operated at a flow rate of 1-8 m³/min.

6. The sterilizer of claim 2, wherein each of the ceramic electrode tubes comprises a ceramic shell, metal powders, and two silicone seals; the ceramic shell comprises aluminum oxide (Al₂O₃) of 99.5% by weight, an outer diameter of the ceramic shell is 15-25 mm, and a wall thickness of the ceramic shell is 1-3 mm; the two silicone seals are disposed on both ends of the ceramic shell, respectively; each silicone seal comprises silicon dioxide (SᵢO₂); and a copper or aluminum wire having a diameter of 2±0.5 mm is disposed through both central points of the two silicone seals.

7. The sterilizer of claim 6, wherein the ceramic shell is filled with the metal powders comprising 30-60 parts of aluminum powder by mass, 30-60 parts of magnesium powder by mass, 10-30 parts of copper powder by mass, and 0.1-5 parts of titanium powder by mass; and the metal powders have a particle size of 80-150 mesh.

8. A method for eliminating a pathogen comprising coronavirus using the sterilizer of claim 1, the method comprising: spraying the disinfecting airflow activated by the cold plasma airflow and the UV light on the surface of the sample contaminated with a pathogen comprising coronavirus for 30-150 seconds; and circulating the disinfecting airflow through the airflow circulation system thus increasing the disinfection efficiency of the sterilizer and reducing ozone emission.

9. The method of claim 8, wherein the cold plasma generator works at an operating voltage of 10-40 kV, an operating frequency is 8-20 kHz, and a power density is 0.5-2.5 W/cm²; the UV light generator emits the UV light at a wavelength of 222±10 nm, and a power density is 0.25-0.65 W/cm².
